(19) United States
(12) Patent Application Publication
(10) Pub. No.: US 2006/0101536 A1
(43) Pub. Date: May 11, 2006

(54) Eucalyptus urophylla transformation and regeneration

(75) Inventors: Shujun Chang, N. Charleston, SC (US); Robert D. Thomas, Summerville, SC (US); Levis W. Handley, Takoma Park, MD (US); Marie B. Connett, Charleston, SC (US); Randy L. Hamilton, Charleston, SC (US)

Correspondence Address: FOLEY AND LARDNER LLP;SUITE 500, 3000 K STREET NW, WASHINGTON, DC, 20007, US

(73) Assignee: ArborGen, LLC

(21) Appl. No.: 10/981,742

(22) Filed: Nov. 5, 2004

(51) Int. Cl. A01H 1/00; C12N 15/82

(52) U.S. Cl. 800/278; 800/294

(57) ABSTRACT

The present invention relates to a method for transforming and selecting plant explants. The transformation method includes pre-culturing the explants in the presence of an Agrobacterium inducer and exposing the transformed explants to a shoot regeneration media that accelerates shoot development. Plants generated from this transformation method are provided. In particular, methods for obtaining transgenic E. urophylla and E. saligna cells and therefrom regenerating stably transformed E. urophylla and E. saligna trees are provided. The invention also provides media, methods, and plasmids for selecting and regenerating plants.

### FIELD OF THE INVENTION

The present invention relates to a method for transforming tree cells with foreign DNA and regenerating stably transformed trees therefrom. In particular, this invention teaches an Agrobacterium-mediated gene transfer method for transforming and regenerating transgenic plants from tree explants. The transformation process described herein utilizes a pre-culture medium that stimulates cell division and a shoot generation medium that accelerates shoot development. Plants generated from this transformation process are provided. In particular, the invention provides methods for increasing transformation efficiency and shoot regeneration from elite and recalcitrant clones. The invention also relates to media, methods, and plasmids for selecting and regenerating plants, particularly forest trees.

### BACKGROUND

Plant genetic engineering has provided great potential for the improvement of commercially important plant species. In recent years, the genetic engineering of trees has gained momentum and finds particular application in the pulping and timber industries. Several established tree transformation systems exist for such species as sweetgum (Sullivan and Lagrinini 1993), European larch (Huang et al. 1991), yellow poplar (Wilde et al. 1992) and many Populus sp. (Minocha et al. 1986, Fillatti et al. 1988, De Block 1990, Brasileiro et al. 1992, Tsai et al. 1994). Species in the Populus genera have served as the model systems for the genetic engineering of trees (Kim et al. 1997). Various traits, such as insect resistance and herbicide tolerance, have been engineered into these tree species (Klopfenstein et al. 1993, De Block 1990). Thus, the potential for genetically engineering tree species is great for commercially important tree species, including Eucalyptus.

Eucalyptus plants comprise more than five hundred species. Eucalyptus has a high growth rate, adapts to wide range of environments, and displays little susceptibility to insect damage. In addition to its exceptional growth properties, Eucalyptus trees provide the largest source of fibers for the paper industry. Fibers from hardwood species, such as Eucalyptus, are generally much shorter than fibers from softwoods, such as pine. The shorter fibers produced from Eucalyptus results in the production of pulp and paper with desirable surface characteristics, including smoothness and brightness, but low tear or tensile strength. As a timber, Eucalyptus provides tall, straight timber with a medium to high density. Eucalyptus timber is general-purpose; finds use in the plywood and particleboard industry, furniture industry; and provides a source of firewood and construction materials.

Most reports demonstrating transformation of Eucalyptus use Eucalyptus seedlings rather than elite genotypes obtained through breeding programs. For example, WO 99/48355 describes a method for transforming young leaf explants from seedlings of E. grandis and E. camaldulensis. Even though the transformation was successful, there are two main problems with the described method. First, the regeneration protocol works for seedling explants, but it does not work with explants from elite genotypes. Second, even with seedling explants and the claimed improvements, the transformation efficiency is limited to 2.2% or lower for cotyledon explants of the two species and the hypocotyl explants of E. camaldulensis. An improved transformation and regeneration protocol was reported for E. camaldulensis seedlings by Ho et al., Plant Cell Reports 17:675-680 (1998); but, the protocol was not repeatable even with E. camaldulensis seedlings. Thousands of explants were cultured, and transgenic callus lines were produced, but the number of shoots recovered was minimal. Harcourt et al. Molecular Breeding 6:306-315 (2000) reported the transformation of E. camaldulensis seedlings with the insecticidal cry3A gene and the herbicide resistant bar gene. Although five herbicide resistant callus lines were regenerated from an unnumbered explants derived from fifty seedlings, one line was difficult to propagate in culture or in the greenhouse, and another line was proven to be an escape. Moreover, the line that was difficult to propagate was one of the two lines that were analyzed at molecular level, and it was the only line with a single insertion. These studies indicate that even when the recovery of transgenic Eucalyptus plants is possible, the low transformation efficiency precludes delivery of a desired gene into many genotypes. Thus, a need continues to exist for a genotype-independent method of Eucalyptus transformation and regeneration.

Although micropropagation of Eucalyptus seedlings has been performed, de novo shoot regeneration has been limited to seedlings instead of the selected or “elite” clones of commercially important Eucalyptus species. Elite genotypes, which arise through successive rounds of breeding, are valued for their combination of economically desirable traits. Unlike seedling transformation, which requires a large number of genotypes to ensure co-segregation of growth traits with the desired trait conferred by transgene expression, the transformation of elite clones would provide an efficient and advantageous system for genetically engineering tree species. Elite genotypes can be selected based on many years of clonal field test with a large number of starting genotypes. Like many other fast growing hardwood tree species, it takes years of field evaluation before relatively accurate predictions of a trait can be made for Eucalyptus. Therefore, if seedlings are used for genetic engineering, an even larger number of genotypes is needed for successful selection of a growth trait together with a desired trait conferred by transgene expression.

Both GB2298205 (WO/9625504) and EP 1050209 claimed the use of 1-(2-chlorl-4-pyridyl)-3-phenylurea or N-(2-chloro-4-pyridyl)-N′-phenylurea (4-PU or 4CPPU) as the primary cytokinin for regeneration of transgenic shoots. In both cases, antibiotics were used as selection agents. WO/9625504 demonstrates the transformation of explants from mature genotypes, but the inventors used seedling explants for E. globulus and E. nitens to demonstrate transformation. EP 1050209 uses a vertical rotary culture system for inducing formation of transgenic primordia. Although transformation was demonstrated with rejuvenated explants from mature trees, the transformation efficiency was calculated based on transgenic callus production and there was no indication of the frequency for transgenic plant production. Since efficient de novo shoot regeneration is critical for genetic engineering, there is a need to develop a highly efficient regeneration system for the selection of clones from commercially important Eucalyptus species.

Accordingly, there is a need to increase the frequency of transforming Eucalyptus cells and regenerating stably transformed plants from clones of recalcitrant species.

Accordingly, there is a need to increase the transformation and regeneration efficiency of E. urophylla and E. saligna recalcitrant to transformation.

### SUMMARY OF THE INVENTION

Contemplated in the present invention is a method for transforming at least one cell of a E. urophylla explant with a foreign DNA, comprising (i) pre-culturing a tree explant on a medium comprising an inducer of Agrobacterium; (ii) exposing said explant to an Agrobacterium strain containing a transformation vector carrying said foreign DNA; (iii) selecting a transformed explant, wherein said foreign DNA is transferred to at least one cell of said transformed explant; and (iv) regenerating said transformed explant to produce a complete plant. Preferably, the inducer of Agrobacterium is acetosyringone and the concentration of said acetosyringone is from about 10 to about 400 mg/l. Also preferred, the concentration of said acetosyringone is from about 5 to about 200 mg/l and the medium further comprises auxin and/or cytokinin.

The invention contemplates a pre-culturing step comprises culturing a plant explant on a nutrient medium before Agrobacterium transformation. The pre-culture medium comprises an inducer of Agrobacterium, such as acetosyringone. In one embodiment, the explant is pre-cultured in the dark from about 1 to about 6 days. Preferably, the explant is pre-cultured for about 4 days. In one embodiment, the pre-culture medium may optionally comprise plant growth regulators, including auxin and cytokinin

In another embodiment, the auxin is selected from the group consisting of NAA, 2,4-D, IBA, and IAA. In one embodiment, the concentration range of any one of NAA, 2,4-D, IBA, and IAA is from about 0.1 to about 10 mg/l. Preferably, the concentration range is from about 0.2 to about 5 mg/l. More preferably, the concentration range is from about 0.2 to about 3 mg/l.

In another embodiment, the cytokinin is selected from the group consisting of zeatin, kinetin, and BA. In one embodiment, the concentration range of any one of said zeatin, kinetin, and BA is from about 0.25 to about 15 mg/l. Preferably, the concentration range is from about 1 to about 10 mg/l. More preferably, the concentration range is from about 1 to about 6 mg/l.

In another embodiment, the explant is at least one of a leaf, a petiole, an internodal tissue, floral tissue, embryogenic tissue, or embryogenic culture.

In one embodiment, the tissues are selected independent of age or developmental stage.

In another embodiment, the method is genotype-independent.

In another embodiment, all of the cells of said transformed explant comprise the foreign DNA.

The invention also contemplates a method for transforming at least one cell of a E. saligna explant with a foreign DNA, comprising (i) culturing a tree explant on a pre-culture medium comprising an inducer of Agrobacterium; (ii) exposing said explant to an Agrobacterium strain containing a transformation vector carrying said foreign DNA; (iii) selecting a transformed explant, wherein said foreign DNA is transferred to at least one cell of said transformed explant; and (iv) regenerating said transformed explant to produce a complete plant.

The invention contemplates a pre-culturing step comprises culturing a plant explant on a nutrient medium before Agrobacterium transformation. The pre-culture medium comprises an inducer of Agrobacterium, such as acetosyringone. In one embodiment, the explant is pre-cultured in the dark from about 1 to about 6 days. Preferably, the explant is pre-cultured for about 4 days. In one embodiment, the pre-culture medium may optionally comprise plant growth regulators, including auxin and cytokinin

In another embodiment, the auxin is selected from the group consisting of NAA, 2,4-D, IBA, and IAA. In one embodiment, the concentration range of any one of NAA, 2,4-D, IBA, and IAA is from about 0.1 to about 10 mg/l. Preferably, the concentration range is from about 0.2 to about 5 mg/l. More preferably, the concentration range is from about 0.2 to about 3 mg/l.

In another embodiment, the cytokinin is selected from the group consisting of zeatin, kinetin, and BA. In one embodiment, the concentration range of any one of said zeatin, kinetin, and BA is from about 0.25 to about 15 mg/l. Preferably, the concentration range is from about 1 to about 10 mg/l. More preferably, the concentration range is from about 1 to about 6 mg/l.

In another embodiment, the explant is at least one of a leaf, a petiole, an internodal tissue, floral tissue, embryogenic tissue, or embryogenic culture.

In one embodiment, the tissues are selected independent of age or developmental stage.

In another embodiment, the method is genotype-independent.

In another embodiment, all of the cells of said transformed explant comprise the foreign DNA.

### BRIEF DESCRIPTION OF THE DRAWINGS

FIG. 1 provides a schematic representation of the vector pWVC20.

FIG. 2 provides a schematic representation of the vector pWVC21.

FIG. 3 provides a schematic representation of the vector pWVC23.

FIG. 4 provides a schematic representation of the vector pWVC24.

FIG. 5a provides a schematic representation of the vector pWVC25.

FIG. 5b provides a schematic representation of the vector pWVC26.

FIG. 6 provides a schematic representation of the vector pWVC30.

FIG. 7 provides a schematic representation of the vector pWVC33.

FIG. 8a provides a schematic representation of the vector pWVC34.

FIG. 8b provides a schematic representation of the vector pWVC35.

### DETAILED DESCRIPTION OF THE INVENTION

The methods of the present invention for tree transformation overcome the low transformation efficiencies obtained from the transformation of elite and recalcitrant genotypes. In its broadest aspect, the methods relate to increasing the efficiencies transformation and shoot regeneration from tree explants of recalcitrant species. In one aspect, the methods provide increase transformation and regeneration efficiencies of recalcitrant species, such as E. urophylla and E. saligna. Increases in transformation efficiency are accomplished by pre-culturing explants on a medium that stimulates cell division. The shoot regeneration frequency of the transformed explants is improved by culturing the explants on a medium that promotes shoot regeneration.

In the description that follows, a number of scientific and technical terms are used extensively. Unless defined otherwise, all technical and scientific terms used herein share the same meaning as commonly understood by one of ordinary skill in the art to which this invention belongs. The following definitions are provided to facilitate understanding of the invention.

Abaxial, as used herein, refers to the lower side of a leaf.

Agrobacterium inducer refers to a molecule that induces expression of Agrobacterium virulence genes that code for products that control excision and delivery of the T-DNA into the host plant nucleus. In the present description, Agrobacterium inducers were added to both the pre-culture medium and the Agrobacterium culture medium. In this invention, Agrobacterium inducers include, but are not limited to, phenolic compounds, such as acetosyringone. Addition of an inducer improves Agrobacterium infection frequency and consistency.

Agrobacterium-mediated transformation is a method by which DNA is stably inserted into the genome of a plant cell through the use of the Ti (tumor-inducing) plasmid from Agrobacterium tumefaciens. A small portion of the Ti plasmid, known as the T-DNA, is incorporated into the nucleus of the host plant cell. Alternatively, the Ri plasmid from Agrobacterium rhizogenes may be used for transformation. In Agrobacterium-mediated transformation, the gene(s) or DNA intended for plant introduction is positioned between the left and right borders of the T-DNA.

Antioxidant, as used herein, refers to a compound that minimizes the exudation of phenolic materials from a plant explant. In the present invention, ascorbic acid may be used as an antioxidant.

In the present description, the term auxin encompasses a class of plant growth regulators that are characterized principally by their capacity to stimulate cell division in excised plant tissues. In addition to their role in cell division and cell elongation, auxins influence other developmental processes, including root initiation. In the present invention, auxin and auxin-type growth regulators include, but are not limited to, naphthaleneacetic acid (NAA), 2,4-Dichlorophenoxy acetic acid (2,4-D), indole-3-butyric acid (IBA), and indole-3-acetic acid (IAA).

Casein hydrolysate-like compound refers to a composition that is structurally and functionally related to casein hydrolysate. In the context of the phrase “casein hydrolysate-like compound, the term denotes a compound that has a similar composition of amino acids as casein hydrolysate, and achieves the same function as casein hydrolysate, but differs in one or more components. For example, a casein hydrolysate-like compound can possess elevated concentrations of amino acids such as glutamine and arginine, important amino acids for growing and maintaining regenerable cells of certain plants in plant tissue culture.

A cloning vector is a genetic element, such as a plasmid, cosmid, or bacteriophage, that has the capability to replicate autonomously in a host cell. Cloning vectors comprise one or a small number of restriction endonuclease recognition sites in which foreign DNA sequences can be inserted in a determinable orientation and a marker gene that encodes a product suitable for the identification and selection of cells transformed with the cloning vector. Marker genes include genes whose products confer antibiotic or herbicide resistance. The insertion of a foreign DNA sequence into a cloning vector does not interfere with an essential biological function of the cloning vector or the marker gene.

Cytokinin refers to a class of plant growth regulators that are characterized by their ability to stimulate cell division and shoot organogenesis in tissue culture. In the present invention, cytokinins include, but are not limited to, N⁶-benzylaminopurine (BAP), N⁶-benzyladenine (BA), zeatin, kinetin, thiadiazuron (TDZ), 2-isopentenyladenine (2ip), and 4-CPPU (N-(2-chloro-4-pyridyl)-N′-phenylurea)).

Derivative refers to a substance that is structurally and functionally related to another substance. In the context of the phrase “derivative of casein hydrolysate,” the term denotes a substance that has a similar composition of amino acids as casein hydrolysate, and achieves the same function as casein hydrolysate, but differs in one or more components. For example, a derivative of casein hydrolysate may have more arginine and/or less valine than a typical casein hydrolysate.

As used herein, elite genotype refers to commercially important genotypes obtained and selected for through successive breeding programs.

The term explant refers to plant tissue that is a target for transformation. Preferred explants comprise leaf, petiole, floral tissue, internodal tissues, and embryogenic tissues harvested from plants grown in vivo and/or in vitro.

The term expression refers to the biosynthesis of a gene product. For example, expression of a gene involves transcription of the DNA sequence into mRNA and translation of the mRNA into one or more polypeptides. The RNA generated may code for a protein or polypeptide or may code for an RNA interfering, or antisense molecule.

An expression vector is a genetic element comprising a gene sequence that is expressed in a host cell. Typically, the expression of the gene sequence is controlled by several regulatory elements, including constitutive and inducible promoters, tissue-preferred regulatory elements, and enhancers. Such a gene is said to be “operably linked” to the regulatory elements.

A foreign DNA is DNA isolated from another species or from the species of interest and re-introduced into the same species. The DNA may be a structural gene, an antisense gene, DNA fragments, etc.

A gene is a heritable DNA sequence that is transcribed into messenger RNA (mRNA) which is then translated into a sequence of amino acids characteristic of a polypeptide.

A non-chimaeric transgenic plant is the product of a transformation event wherein essentially all of the cells are transformed and a foreign DNA is transferred to progeny.

Operably linked describes combining two or more molecules in such a fashion that in combination they function properly in a plant cell. For instance, a promoter is operably linked to a structural gene when the promoter controls transcription of the structural gene.

As used herein, plant is any of various photosynthetic, eukaryotic, multicellular organisms of the kingdom Plantae characteristically producing embryos, containing chloroplasts, and having cellulose cell walls. As part of a plant, a plant tissue may be treated according to the methods of the present invention to produce a transgenic plant. Many suitable plant tissues can be transformed according to the present invention and include, but are not limited to, somatic embryos, pollen, leaves, stems, calli, stolons, microtubers, and shoots. Thus, the present invention envisions the transformation of angiosperm and gymnosperm plants such as turfgrass, wheat, maize, rice, barley, oat, sugar beet, potato, tomato, tobacco, alfalfa, lettuce, carrot, strawberry, cassava, sweet potato, geranium, soybean, oak, pine, fir, acacia, eucalyptus, walnut, and palm. According to the present invention plant tissue also encompasses plant cells. Plant cells include suspension cultures, callus, embryos, meristematic regions, callus tissue, leaves, roots, shoots, gametophytes, sporophytes, pollen, seeds and microspores. Plant tissues may be at various stages of maturity and may be grown in liquid or solid culture, or in soil or suitable media in pots, greenhouses or fields. A plant tissue also refers to any clone of such a plant, seed, progeny, propagule whether generated sexually or asexually, and descendents of any of these, such as cuttings or seed. Of particular interest are conifers such as pine, fir and spruce, monocots such as Kentucky bluegrass, creeping bentgrass, maize, and wheat, and dicots such as Eucalyptus, Acacia, aspen, Sweetgum, poplar, cotton, tomato, lettuce, Arabidopsis, tobacco, and geranium.

Plant promoter is a promoter capable of initiating transcription in plant cells whether or not its origin is a plant cell. Exemplary plant promoters include, but are not limited to, those that are obtained from plants, plant viruses, and bacteria such as Agrobacterium or Rhizobium which comprise genes expressed in plant cells. Examples of promoters under developmental control include promoters that preferentially initiate transcription in certain tissues, such as leaves, roots, or seeds. Such promoters are referred to as tissue-preferred promoters. Promoters which initiate transcription only in certain tissue are referred to as tissue-specific promoters. A cell type-specific promoter primarily drives expression in certain cell types in one or more organs, for example, vascular cells in roots or leaves. An inducible or repressible promoter is a promoter which is under environmental control. Examples of environmental conditions that may effect transcription by inducible promoters include anaerobic conditions or the presence of light. Tissue specific, tissue preferred, cell type specific, and inducible promoters constitute the class of non-constitutive promoters. A constitutive promoter is a promoter which is active under most environmental conditions, and in most plant parts.

Pre-culture medium, as used herein, is the nutrient medium upon which plant explants are cultured prior to transformation with Agrobacterium and is needed for increasing transformation efficiency and plant regeneration. The pre-culture medium comprises an inducer of Agrobacterium, such as acetosyringone. The pre-culture medium may optionally comprise plant growth regulators, including auxin and cytokinin.

Progeny: a “progeny” of the present invention, such as the progeny of a transgenic plant, is one that is born of, begotten by, or derived from a plant or the transgenic plant. Thus, a “progeny” plant, i.e., an “F1” generation plant is an offspring or a descendant of the transgenic plant produced by the inventive methods. A progeny of a transgenic plant may contain in at least one, some, or all of its cell genomes, the desired polynucleotide that was integrated into a cell of the parent transgenic plant by the methods described herein. Thus, the desired polynucleotide is “transmitted” or “inherited” by the progeny plant. The desired polynucleotide that is so inherited in the progeny plant may reside within a T-DNA construct, which also is inherited by the progeny plant from its parent. The term “progeny” as used herein, also may be considered to be the offspring or descendants of a group of plants.

Shoot regeneration medium is the inventive medium designed to regenerate transgenic shoots. The shoot regeneration medium comprises inorganic salts, a mixture of amino acids and vitamins, an antioxidant, organic nitrogen, and plant growth regulators.

Somatic embryogenesis is a method of clonal propagation wherein the embryo develops from vegetative or somatic tissue rather than as a product of gametic fusion.

Stably transformed refers to a transgenic plant that is capable of transmitting foreign DNA to progeny.

Structural gene is a DNA sequence that is transcribed into messenger RNA (mRNA) which is then translated into a sequence of amino acids characteristic of a specific polypeptide.

Substantially free refers to the relative absence of a first compound from a second compound. The term denotes that less than 10%, 5%, 4%, 3%, 2%, 1% or even 0% of a first compound can be detected in a second compound.

A transgenic plant is a plant comprising foreign DNA. In this invention, a transgenic plant is derived from Agrobacterium-mediated transformation. Preferably, the transgenic plant is fertile and capable of transmitting the foreign DNA to progeny plant through sexual reproduction.

Transcription and translation terminators: The expression DNA constructs of the present invention typically have a transcriptional termination region at the opposite end from the transcription initiation regulatory region. The transcriptional termination region may be selected, for stability of the mRNA to enhance expression and/or for the addition of polyadenylation tails added to the gene transcription product.

Tree, as used herein, refers to any perennial vegetation that accumulates a wood core. Trees include angiosperms and gymnosperm species. Examples of trees include poplar, and hardwoods such as Eucalyptus, Douglas fir, pine, nut trees, e.g., walnut and almond, fruit trees, e.g., apple, plum, citrus and apricot, and hardwood trees, such as ash, birch, oak, and teak. Of particular interest are conifers such as pine, fir, spruce, Eucalyptus, Acacia, aspen, Sweetgum, and poplar.

The present invention provides genotype-independent methods for transforming tree explants and generating transgenic progeny therefrom. The methods of the present invention contemplate pre-culturing tree explants in the presence of an Agrobacterium inducer. The methods of the present invention further envision culturing the transformed explants on a shoot regeneration medium comprising amino acids, vitamins, plant growth regulators, glucose, and an antioxidant.

The methods of the instant invention provide a genotype-independent method of Eucalyptus explant transformation and shoot regeneration. Any Eucalyptus explant may be transformed by the methods of the instant invention, including Eucalyptus trees grown in natural environments and Eucalyptus explants clonally propagated. The explant may be selected from any Eucalyptus species, including Eucalyptus alba, Eucalyptus bancroftii, Eucalyptus botryoides, Eucalyptus bridgesiana, Eucalyptus calophylla, Eucalyptus camaldulensis, Eucalyptus citriodora, Eucalyptus cladocalyx, Eucalyptus coccifera, Eucalyptus curtisii, Eucalyptus dalrympleana, Eucalyptus deglupta, Eucalyptus delagatensis, Eucalyptus diversicolor, Eucalyptus dunnii, Eucalyptus ficifolia, Eucalyptus globulus, Eucalyptus gomphocephala, Eucalyptus gunnii, Eucalyptus henryi, Eucalyptus laevopinea, Eucalyptus macarthurii, Eucalyptus macrorhyncha, Eucalyptus maculata, Eucalyptus marginata, Eucalyptus megacarpa, Eucalyptus melliodora, Eucalyptus nicholii, Eucalyptus nitens, Eucalyptus nova-angelica, Eucalyptus obliqua, Eucalyptus occidentalis, Eucalyptus obtusiflora, Eucalyptus oreades, Eucalyptus pauciflora, Eucalyptus polybractea, Eucalyptus regnans, Eucalyptus resinifera, Eucalyptus robusta, Eucalyptus rudis, Eucalyptus saligna, Eucalyptus sideroxylon, Eucalyptus stuartiana, Eucalyptus tereticornis, Eucalyptus torelliana, Eucalyptus urnigera, Eucalyptus urophylla, Eucalyptus viminalis, Eucalyptus viridis, Eucalyptus wandoo, and Eucalyptus youmanni.

The methods of the instant invention contemplate the transformation of Eucalyptus explants obtained from a stock culture of elite Eucalyptus genotypes. Micropropagated shoot cultures may be generated by harvesting newly flushed apical or axillary shoots and surface sterilizing the tissues in a sterilization solution. Sterilization solutions, such as 1-5% bleach solution, are known in the art and repeated rinsing with sterile, distilled water can be performed. Eucalyptus stock cultures can be maintained as shoot clusters on a maintenance medium comprising inorganic salts, carbon sources, vitamins, and cytokinins. In the present invention, stock cultures are maintained on Eucalyptus Maintenance (EM) medium (Table 1) comprising Woody Plant Medium (WPM) salts (Loyd and McCown, 1980) and N⁶-benzyladenine (BA). Alternatively, other salt media, such as MS medium (Murashige and Skoog 1962) or Lepoivre medium, may be used.

TABLE 1
Eucalyptus Maintenance Medium (EM medium)

| Medium | Amount per Liter | |
|---|---|---|
| Components | of Medium | |
| WPM salts | 1 | package (Sigma) |
| Ca(NO₃)₂.4H₂O | 3.7 | g |
| MgSO₄.4H₂O | 0.37 | g |
| Nicotinic Acid | 0.5 | mg |
| Thiamine.HCl | 0.5 | mg |
| Pyridoxin.HCl | 0.5 | mg |
| D-Pantothenic Acid | 1.0 | mg |
| Myo-inositol | 0.1 | g |
| BA | 0.1-1 | mg |
| Bacto-agar | 5-8 | g |

The present invention provides a method of genotype-independent transformation. The methods of the present invention teach the transformation of explants independent of age and developmental stage. Tree explants obtained from the stock culture can be used for transformation. Tree explants may be selected from one of more of leaf, petiole, internodal, and floral tissues. In the present invention, leaf explants are selected due to their abundant supply and ease of transformation. The tip portion of leaves may be removed or punctured with a forcep to increase the number of wounded cells. In the instant application, leaf explants are placed on the pre-culture medium abaxial side down.

In the instant application, plant explants are cultured on a pre-culture medium. A pre-culture medium is a nutrient medium upon which plant explants are cultured before Agrobacterium transformation. Specifically, the inventive pre-culture medium increases transformation efficiency and plant regeneration. The pre-culture medium comprises an inducer of Agrobacterium, such as acetosyringone. Alternatively, other Agrobacterium inducers may be used, such as hydroxyphenylpropanoids, phenolic compounds, and coniferin. Woody Plant Medium (WPM) salts (Loyd and McCown, 1980) were used in the present invention; however, other salt media, such as MS medium (Murashige and Skoog 1962) or Lepoivre medium, may be used. Optionally, the pre-culture medium may comprise plant growth regulators, including auxin and cytokinin. In the present invention, plant explants were pre-cultured for four days in the dark on the Pre-Culture Medium of Table 2. Other pre-culture media and time periods of culture may be used.

TABLE 2
Plant Pre-Culture Medium

| Medium | Amount per Liter | |
|---|---|---|
| Components | of Medium | |
| WPM salts | 1 | package (Sigma) |
| Ca(NO₃)₂.4H₂O | 3.7 | g |
| MgSO₄.4H₂O | 0.37 | g |
| Nicotinic Acid | 0.5 | mg |
| Thiamine.HCl | 0.5 | mg |
| Pyridoxin.HCl | 0.5 | mg |
| D-Pantothenic Acid | 1.0 | mg |
| Myo-inositol | 0.1 | g |
| BA | 0.1-1 | mg |
| Bacto-agar | 5-8 | g |
| Acetosyringone | 5-200 | mg |
| NAA | 0.2-3 | mg |
| Zeatin | 1-6 | mg |

The methods of the present invention teach pre-culturing the explants on a pre-culture medium containing a high concentration of auxin or auxin-type growth regulators. Preferably, the auxin or an auxin-type growth regulator is selected from the group consisting of naphthaleneacetic acid (NAA), 2,4-Dichlorophenoxyacetic acid (2,4-D), indole-3-butyric acid (IBA), and indole-3-acetic acid (IAA). More preferably the auxin is NAA. The concentration range of said auxin is from about 0.1 to about 10 mg/l. The preferred auxin concentration is from about 0.2 to about 5 mg/l. More preferably, the auxin concentration is from about 0.2 to about 3 mg/l.

Preferably, the methods of the present invention provide for pre-culturing tree explants on a pre-culture medium comprising sufficient cytokinin. The cytokinin is selected from the group consisting of N⁶-benzylaminopurine (BAP), N⁶-benzyladenine (BA), zeatin, kinetin, 4-CPPU (N-(2-chloro-4-pyridyl)-N′-phenylurea)), thiadiazuron (TDZ), and 2-isopentenyladenine (2ip). The concentration range of said cytokinin is from about 0.25 to about 15 mg/l. Preferably, the cytokinin concentration range is from about 1 to about 10 mg/l. More preferably, the cytokinin concentration range is from about 1 to about 6 mg/l.

The methods of the present invention provide for pre-culturing tree explants on a pre-culture medium comprising an Agrobacterium inducer. The inducer may be acetosyringone. The concentration range of acetosyringone is from about 10 to about 400 mg/l. Preferably, the concentration range is from about 5 to about 200 mg/l.

The present invention contemplates the method of optionally pre-culturing tree explants on a pre-culture medium comprising both an auxin and an Agrobacterium inducer. The invention demonstrates that the combination of an auxin with an Agrobacterium inducer results in higher infection rates than either component independently. The explants can be pre-cultured on said pre-culture medium for about 1 to about 6 days prior to the introduction of Agrobacterium. Preferably, the explants are pre-cultured for about 4 days. The pre-culturing step can occur under both dark and light conditions; however, it is preferable to culture in the dark.

Eucalyptus explant transformation is performed with different strains of A. tumefaciens harboring a transformation vector. One such vector is GV2260, comprising a GUS gene operably linked to a promoter, such as an actin promoter or a constitutive promoter. The vector will also carry a herbicide resistance gene operably linked to a promoter. In the present invention, the acetolactate synthase (ALS) gene confers herbicide resistance and is driven by its Arabidopsis native promoter. See U.S. Pat. No. 6,225,105. An A. tumefaciens culture suspended in induction medium (AIM formulation) is dripped by pipette on to the explants such that all cut edges are exposed to the bacteria. Alternatively, the explants may be transformed by vacuum infiltration, floral dip, and other methods of Agrobacterium-mediated transformation that are well known in the art. For a review of Agrobacterium-mediated transformation, see Gelvin, S B. Microbiol. Mol Biol Rev 67:1:16-37 (2003). Upon introduction of Agrobacterium, the explants are co-cultivated with the Agrobacterium for about 3 days on the same medium. Following co-cultivation, the explants are transferred to a shoot regeneration medium for the recovery of transgenic shoots.

The present invention teaches methods for shoot regeneration wherein transformed explants are cultured on a medium comprising a mixture of amino acids and vitamins, plant growth regulators, glucose, and an antioxidant. One such complete medium formulation is listed in Table 3 and is called Euc Regeneration medium. An antioxidant, such as ascorbic acid, may be added to the Euc Regeneration medium to minimize the exudation of plant phenolic compounds. Glucose and glutamine may be added to the medium to accelerate shoot regeneration. Antibiotics, such as carbenicillin, cefotaxime, and timentin can also be included in the medium to prevent bacterial overgrowth. Timentin is the preferred antibiotic. The antibiotic concentration ranges from about 75 to 800 mg/l. Preferably, the antibiotic concentration is about 400 mg/l.

The Euc Regeneration medium contains a mixture of amino acids that do not interfere with amino acid biosynthesis. In the present invention, the Euc Regeneration medium does not interfere with sulfonylurea-based herbicide selection. Preferably, the Euc Regeneration medium does not have branched chain amino acids (e.g. leucine, isoleucine, and valine). More preferably, the media has an amino acid mixture that replaces casein hydrolysate. Most preferably, the amino acid mixture has amino acids important for plant tissue culture growth.

TABLE 3
Eucalyptus Regeneration Medium

| Components for 1 Liter | |
|---|---|
| of Medium | Grams |
| KNO₃ | 1 |
| NH₄H₂PO₄ | 0.25 |
| MgSO₄.7H₂O | 0.25 |
| CaCl₂.2H₂O | 0.10 |
| FeSO₄.7H₂O | 0.0139 |
| Na₂EDTA.2H₂O | 0.01865 |
| MES (Duchefa m1501) | 600.0 |
| MS Micro (½ strength) | |
| MnSO₄.H₂O | 0.00845 |
| ZnSO₄.7H₂O | 0.0043 |
| CuSO₄.5H₂O | 0.0000125 |
| CoCl₂.6H₂O | 0.0000125 |
| KI | 0.000415 |
| H₃BO₃ | 0.0031 |
| Na₂MoO₄.2H₂O | 0.000125 |
| Plant Growth Regulators | |
| Zeatin | |
| NAA (naphthalene acetic acid) | |
| Sugars | |
| Glucose/Sucrose | 20.0 |
| Myo-inositol | 0.100 |
| Amino acid and vitamine mix | |
| Nicotinic Acid | 0.010 |
| Thiamine | 0.010 |
| Ca Pantothenate | 0.001 |
| Pyridoxine | 0.001 |
| Biotin | 0.00001 |
| Ascorbic Acid | 0.050 |
| L-glutamine | 0.1 |
| Arginine | 0.0258 |
| Glycine | 0.00199 |
| Lysine | 0.0508 |
| Methionine | 0.0132 |
| Phenylalanine | 0.0257 |
| Serine | 0.00904 |
| Threonine | 0.00852 |
| Tryptophan | 0.0122 |
| Tyrosine | 0.0127 |
| Gelling Agent | |
| Gelrite | 3.0 |

In the present invention, there is about a 4-day recovery period before the selection of transformed explants. To select for transformed explants, any selectable marker is added to the regeneration medium. Selectable markers include herbicides and antibiotics. Additionally, any screenable marker may be used to select a transformed plant. Examples of screenable markers include B-glucuronidase (GUS), green fluorescent protein (GFP), and luciferase. In the present invention, an herbicide selection agent is used. While the herbicide of the present invention is ALLY, any herbicide may be used. Other herbicides include Oust and Liberty. The herbicide concentration may vary depending on the sensitivity of the explants from a specific species. The selected explants are subcultured every two to three weeks until the formation of adventitious buds. Transformed adventitious shoots are separated from shoot clumps and are stained for GUS (B-glucuronidase) expression. Jefferson et al. EMBO: 6:13:901-3907 (1987). A reporter gene assay, such as GUS, is used to determine transformation efficiency and to ensure that the transformed shoots are not escapes or chimeras.

Upon confirmation of transformation via expression of a screenable marker gene, Southern blot analysis, PCR analysis, or other method known in the art, the transformed shoots are preferably transferred to a medium for shoot elongation. The present invention contemplates a shoot elongation medium comprising MS salts, sucrose, auxin, and giberellic acid. NAA is the preferred auxin and GA3 is the preferred giberellic acid. The shoots are cultured on the shoot elongation medium for about 10 to about 14 days, preferably under dark conditions. For the elongation of E. dunnii clones, additional auxin needs to be added to the elongation media and the shoots should be cultured in the dark for the duration of the elongation.

Following shoot elongation, the shoots are excised and transferred to a root induction medium. Depending on the light conditions, it may be necessary to add plant growth regulators to the root induction medium. The methods of the present invention teach shoot excision at the node or immediately below the node. More preferably, the shoots are excised from a node near the shoot apex. One such rooting medium (Table 4) is comprised of BTM-1 nutrients (Chalupa 1988), activated carbon (MeadWestvaco, Nuchar), and an extra amount of CaCl₂. Alternatively, other low-salt media may be used for the root induction medium, such as Woody Plant Medium or ½ strength MS.

Depending on the light conditions, the rooting media may contain growth regulators to induce root formation. For example, under dark conditions that induce etiolation and auxin production in the shoot apical meristem, it may not be necessary to include auxin in the rooting medium. Additionally, if the shoots are excised in the dark, it may not be necessary to limit the origin of excision to the nodal regions and/or the shoot apex. Alternatively, if the rooting step occurs under light, it may be necessary to include auxin in the rooting medium. Furthermore, if the rooting step occurs in the light, it is preferable to excise the shoots at a node near the shoot apical meristem.

TABLE 4
Preferred rooting medium for Eucalyptus

| BTM-1 Media Components | mg/L |
|---|---|
| NH₄NO₃ | 412 |
| KNO₃ | 475 |
| Ca(NO₃)₂.4H₂O | 640 |
| CaCl₂.2H₂O | 440* |
| MgSO₄.7H₂O | 370 |
| KH₂PO₄ | 170 |
| MnSO₄.H₂O | 2.3 |
| ZnSO₄.7H₂O | 8.6 |
| CuSO₄.5H₂O | 0.25 |
| CoCl₂.6H₂O | 0.02 |
| KI | 0.15 |
| H₃BO₃ | 6.2 |
| Na₂MoO₄.2H₂O | 0.25 |
| FeSO₄.7H₂O | 27.8 |
| Na₂EDTA.2H₂O | 37.3 |
| Myo-inositol | 100 |
| Nicotinic acid | 0.5 |
| Pyridoxine HCl | 0.5 |
| Thiamine HCl | 1 |
| Glycine | 2 |
| Sucrose | 20000 |
| Activated Carbon | 5000 |

*Additional 400 mg/l CaCl₂.2H₂O is added to the medium

For species that are difficult to root, excised shoots can be pulse-treated with a medium having low levels of auxin to induce shoot formation. For example, a medium comprising 0.25 mg/l 2,4-D may be used for pulse treatment. Preferably, the shoots are pulse-treated for about 5-14 days before transferring to BTM-1 medium having activated carbon.

The transformation method of the present invention can be used for introducing any foreign DNA into a Eucalyptus species. Using the methods of the instant invention, any foreign DNA can be stably integrated into a plant cell and transmitted to progeny. For example, a gene involved in lignin biosynthesis, floral development, cellulose synthesis, nutrient uptake and transport, disease resistance, or enhanced resistance to an environmental condition can be introduced into a plant cell by the instant methods.

The methods of the present invention can be used for reducing gene expression in a Eucalyptus species. Reduction of gene expression can be obtained by methods known in the art, including antisense suppression, co-suppression (sense suppression), and double stranded RNA interference. For a general review of gene suppression techniques, see Science, 288:1370-1372 (2000). Exemplary gene silencing methods are also provided in WO 99/49029 and WO 99/53050.

For antisense suppression, a cDNA sequence is arranged in a reverse orientation relative to the promoter sequence in a DNA construct. The cDNA sequence need not be full length relative to either the primary transcription product or fully processed mRNA. Generally, higher identity can be used to compensate for the use of a shorter sequence. Furthermore, the introduced sequence need not have the same intron or exon pattern, and identity of non-coding segments may be equally effective. Normally, a sequence of between about 30 or 40 nucleotides and about full length nucleotides should be used, though a sequence of at least about 100 nucleotides is preferred, a sequence of at least about 200 nucleotides is more preferred, and a sequence of about 500 to about 3500 nucleotides is especially preferred. The nucleic acid segment to be introduced generally will be substantially identical to at least a portion of the endogenous gene or genes to be repressed. The sequence, however, need not be perfectly identical to inhibit expression. The vectors of the present invention can be designed such that the inhibitory effect applies to other genes within a family of genes exhibiting identity or substantial identity to the target gene.

Another well known method of gene suppression in plants in sense co-suppression. Introduction of a nucleic acid sequence configure in the sense orientation provides an effective means by which to block the transcription of target genes. See, Assaad et al. Plant Mol. Bio. 22: 1067-1085 (1993); Flavell Proc. Natl. Acad. Sci. USA 91: 3490-3496 (1994); Stam et al. Annals Bot. 79: 3-12 (1997); Napoli et al., The Plant Cell 2:279-289 (1990); and U.S. Pat. Nos. 5,034,323, 5,231,020, and 5,283,184. For co-suppression, the introduced sequence need not be full length, relative to either the primary transcription product or fully processed mRNA. Preferably, the introduced sequence is not full length, so as to avoid a concurrent sense overexpression phenotype. In general, a higher identity in a shorter than full length sequence compensates for a longer, less identical sequence.

Using the methods of the instant invention, antisense suppression of a gene involved in lignin biosynthesis can be used to modify lignin content and/or composition in a transformed Eucalyptus plant. It has been demonstrated that antisense expression of sequences encoding cinnamyl alcohol dehydrogenase (CAD) in poplar, N. tabacum, and pine leads to the production of lignin having a modified monomeric composition. Grand et al., Planta 163: 232-37 (1985), Yahiaoui et al., Phytochemistry 49: 295-306 (1998), and Baucher et al., Plant Physiol. 112: 1479 (1996), respectively. Accordingly, the methods of the present invention can be used to stably transform and regenerate Eucalyptus species having antisense suppression of a foreign DNA involved in lignin biosynthesis.

The methods of the present invention can be used for regulating floral development in a Eucalyptus species. Several gene products have been identified as critical components for anther development and pollen formation. For example, premature degradation of callose, which is essential for the formation of the microspore cell wall and microspore release, is sufficient to cause male sterility. Worrall et al., Plant Cell 4:7:759-71 (1992). Accordingly, several methods have been developed for producing male sterile plants. U.S. Pat. No. 5,962,769, for example, describes transgenic plants rendered male sterile via expression of avidin. Avidin can be expressed constitutively, in a non-tissue specific manner, or in anther-specific tissues. In addition, male sterility can be induced by antisense suppression of chalcone synthase A gene. van der Meer et al., The Plant Cell 4:253 (1992). By the methods of the present invention, male sterile Eucalyptus and pine species can be produced and regenerated.

The present invention provides plant media comprising a sulfonylurea or related herbicide and a derivative of casein hydrolysate, wherein the derivative is substantially free of branched chain amino acids. Casein hydrolysate, also known as casein powder or casamino acids, is a mixture of amino acids and short peptide fragments obtained from the hydrolysis of casein. While various amino acid compositions are reported in the literature, and various amino acid compositions may be obtained from various sources of casein and various means of hydrolysis, casein hydrolysate obtained from any source by any means normally comprises branched chain amino acids including valine, leucine and isoleucine, aromatic amino acids such as tryptophan, and other amino acids such as glycine. Additionally, while the composition varies, practitioners agree that the compound is critical for initiating growth of plant cells, particularly many types of embryogenic cultures and conifer plant cells, in vitro. Supplementation of the inorganic media components with reduced nitrogen components such as amino acids, and in particular casein hydrolysate, is viewed by those skilled in the art of woody plant tissue culture, and particularly conifer tissue culture, as an essential component to induce or maintain the growth of cultures that can subsequently be regenerated into plants. For example, the initiation, induction and/or maintenance media of embryogenic cell culture of conifer species routinely comprise casein hydrolysate. See e.g. U.S. Pat. Nos. 5,034,326, 5,036,007, 5,041,382, 5,236,841, 5,310,672, 5,491,090, 5,413,930, 5,506,136, 5,534,433, 5,534,434, 5,563,061, 5,610,051, 5,821,126, 5,850,032, 6,200,809 and 6,518,485.

Thus, regardless of whether the media of subsequent culturing steps comprises casein hydrolysate, small amounts of the compound persist. Such trace amounts can wreak havoc during selection. For example, minute quantities of casein hydrolysate can inhibit the effectiveness of selection agents such as sulfonylureas, imidazolinones and triazolopyrimidines, which work to identify transgenic plant cells following transfection by a vector of interest.

As noted above, these selection agents target acetolactate synthase (ALS), which catalyzes the first common step in a plant's biosynthetic pathway of the branched-chain amino acids valine, leucine and isoleucine. Mutant forms of ALS which are resistant to these agents are utilized as selectable markers in recombinant DNA constructs and are paired with selection agents to identify transformed plant cells.

Therefore, minute quantities of casein hydrolysate in the selection medium enable untransformed cells to grow in the presence of sulfonylureas, imidazolinones or triazolopyrimidines. These false positives impede the production of transgenic plants since more samples have to be evaluated in order to identify desired transformants.

The present invention eliminates this problem by modifying the composition of casein hydrolysate so as to be substantially free of branched-chain amino acids. In another embodiment, the derivative of casein hydrolysate comprises a higher percentage of amino acids that are important in plant tissue culture growth, such as arginine or glutamine.

In another aspect, the invention provides a media composition for selecting transgenic plants comprising a tryptophan analog, wherein the media is substantially free of tryptophan.

Feedback-insensitive forms of the AS make useful selectable markers for plant transformations. These markers work in conjunction with tryptophan analogs to identify transformants. ASA2 is a preferred selectable marker.

Tryptophan appears routinely in plant media as a component of casein hydrolysate. The presence of minute concentrations of tryptophan in selection media can allow non-transformants to escape the selection process, thereby yielding false positives.

It was discovered that selecting transformants on media substantially free of tryptophan enhanced the selection process by reducing the number of false positives. Thus, one aspect of the present invention provides a method of selecting a transformed plant cell comprising transforming a plant cell with a vector comprising a gene of interest and a gene encoding a feedback-insensitive form of anthranilate synthase (AS), and growing the transformed cell on a media composition comprising a tryptophan analog, wherein the media is substantially free of tryptophan.

The methods, media and plasmids herein described are useful in selection of transgenic forest tree plants or lines using selection agents that alter amino acid metabolism, such as sulfonylurea and imidazolinone herbicides and methylated tryptophan analogs. They are further useful in selection, pre-selection and pre-transformation media used to culture plant material to be subjected to transformation with selectable markers that alter amino acid metabolism.

The methods, media and plasmids described above are further useful in the regeneration of plants from cell lines that have been selected using selection agents that alter amino acid metabolism. They can be for obtaining plants transformed with selectable markers that alter amino acid metabolism and that also may be resistant to herbicides that alter amino acid metabolism. Accordingly, the inventions herein also are useful for obtaining herbicide-resistant treestocks. In addition, the inventions are useful in providing crop material suitable for herbicide management of weeds in forestry plantings.

The inventive methods, media and plasmids can be used in the selection and regeneration of plants transformed with genes that result in overproduction of specific amino acids such as tryptophan. Accordingly, they may be further useful for obtaining treestocks showing increased or altered growth as a result of such overproduction.

The methods described herein are generally useful for developing and testing new selection media and selective agent doses. The same principles used to design these media can be applied to the design of selection media for positive selection methods, such as the use of normally non-metabolized sugars.

The inventive methods also are useful for developing formulations of amino acid mixtures to supplement plant tissue culture media.

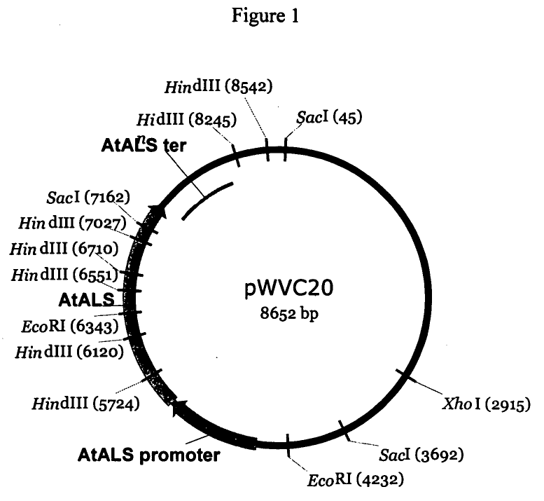

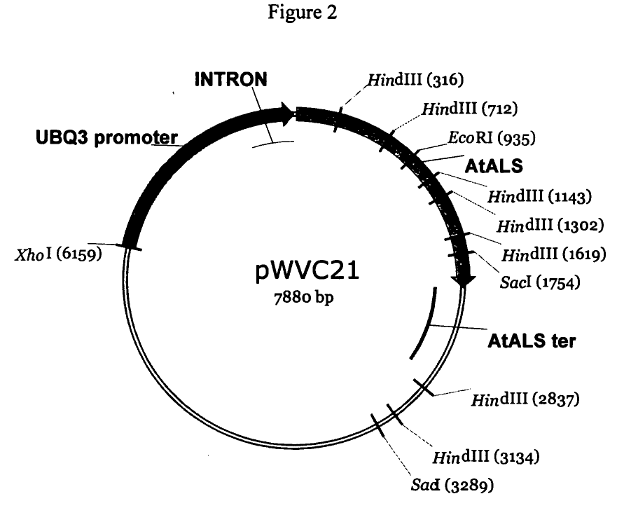

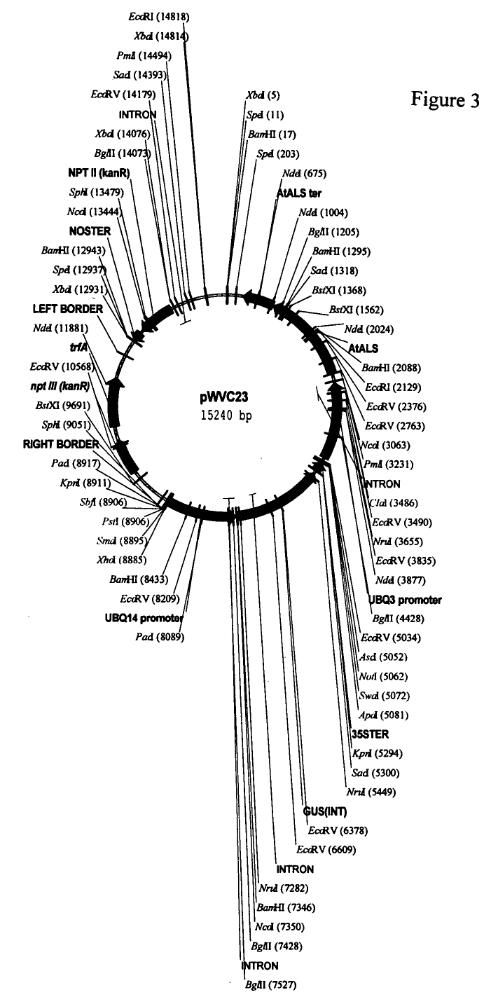

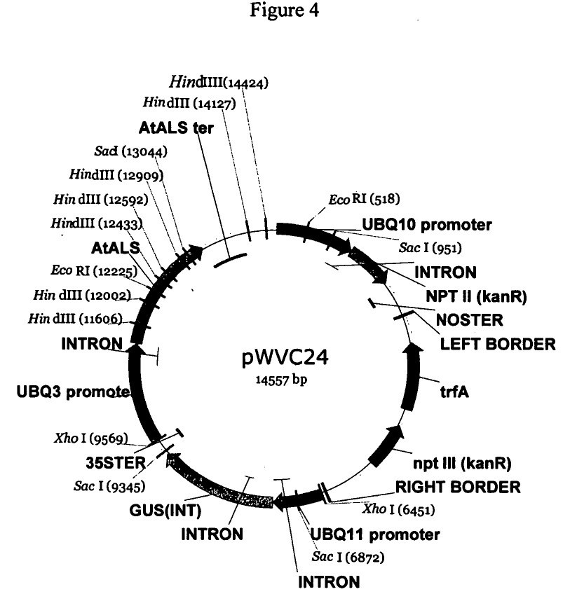

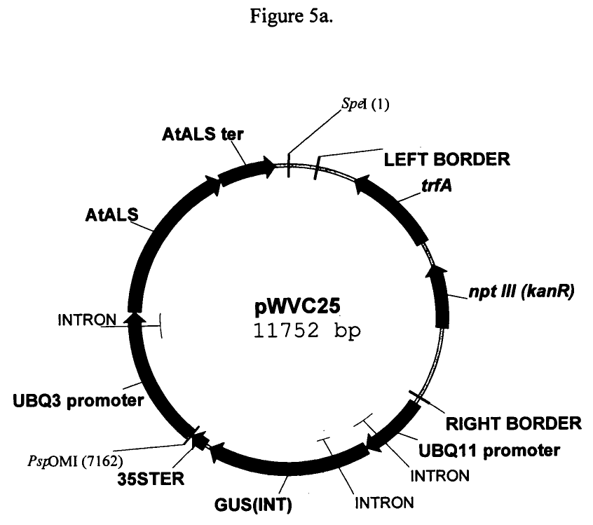

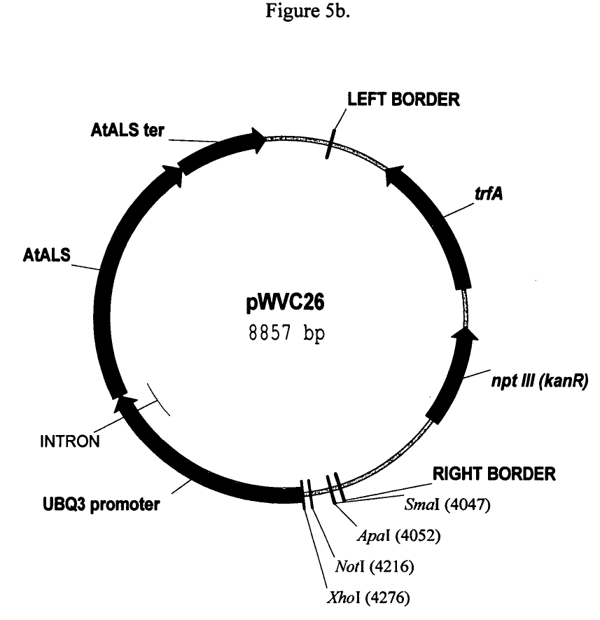

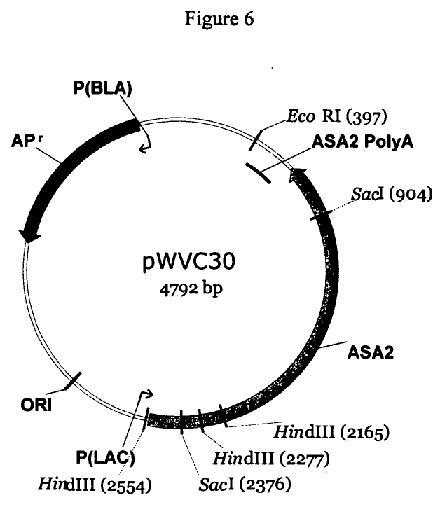

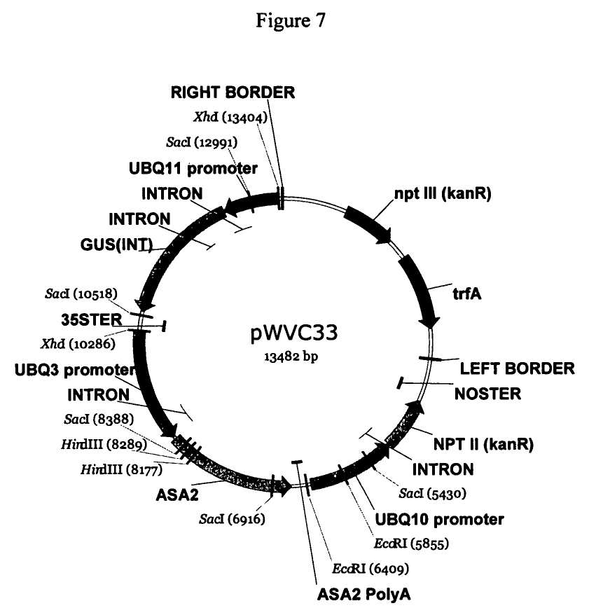

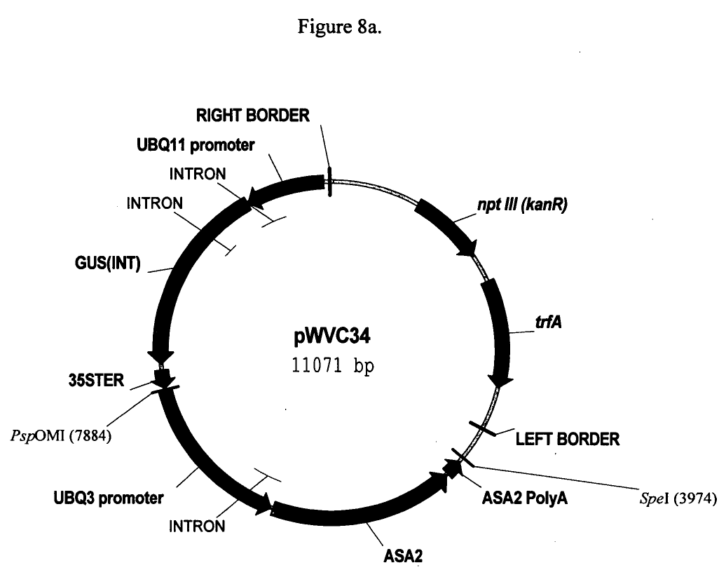

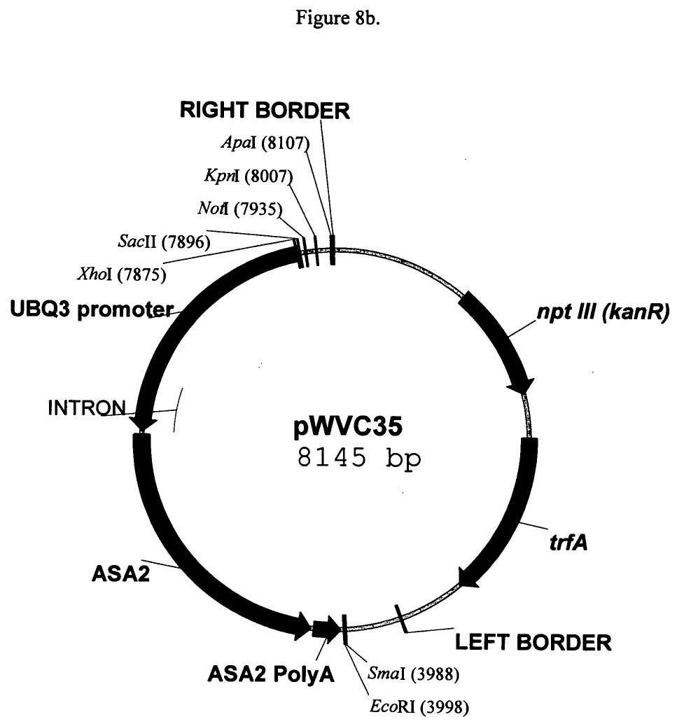

In another embodiment, the invention provides recombinant constructs useful for expressing heterologous proteins in plants. Examples of the inventive vectors include the following: pWVC20 (FIG. 1), pWVC21 (FIG. 2), pWVC23 (FIG. 3), pWVC24 (FIG. 4), pWVC25 (FIG. 5a), pWVC26 (FIG. 5b), pWVC30 (FIG. 6), pWVC33 (FIG. 7), pWVC34 (FIG. 8a), and pWVC35 (FIG. 8b).

Another aspect of the invention provides methods of obtaining wood, wood pulp, paper, resins and essential oils from a plant transformed and selected by the methods of the present invention. Methods for transforming and selecting a transgenic plant are provided above and are known in the art. A transformed plant can be cultured or grown under any suitable conditions. For example, pine can be cultured and grown as described in U.S. Patent Application Publication No. 2002/0100083. Eucalyptus can be cultured and grown as in, for example, Rydelius, et al., Growing Eucalyptus for Pulp and Energy, presented at the Mechanization in Short Rotation, Intensive Culture Forestry Conference, Mobile, Ala., 1994. Wood, wood pulp, paper, resins and essential oils can be obtained from the plant by any means known in the art.

As noted above, the wood and wood pulp obtained in accordance with this invention may demonstrate improved characteristics including, but not limited to any one or more of lignin composition, lignin structure, wood composition, cellulose polymerization, fiber dimensions, ratio of fibers to other plant components, plant cell division, plant cell development, number of cells per unit area, cell size, cell shape, cell wall composition, rate of wood formation, aesthetic appearance of wood, formation of stem defects, rate of growth, rate of root formation ratio of root to branch vegetative development, leaf area index, and leaf shape include increased or decreased lignin content, increased accessibility of lignin to chemical treatments, improved reactivity of lignin, increased or decreased cellulose content increased dimensional stability, increased tensile strength, increased shear strength, increased compression strength, increased shock resistance, increased stiffness, increased or decreased hardness, decreased spirality, decreased shrinkage, and differences in weight, density, and specific gravity.

Phenotype can be assessed by any suitable means. The plants can be evaluated based on their general morphology. Transgenic plants can be observed with the naked eye, can be weighed and their height measured. The plant can be examined by isolating individual layers of plant tissue, namely phloem and cambium, which is further sectioned into meristematic cells, early expansion, late expansion, secondary wall formation, and late cell maturation. See, e.g., Hertzberg, supra. The plants also can be assessed using microscopic analysis or chemical analysis.

Microscopic analysis includes examining cell types, stage of development, and stain uptake by tissues and cells. Fiber morphology, such as fiber wall thickness and microfibril angle of wood pulp fibers can be observed using, for example, microscopic transmission ellipsometry. See Ye and Sundström, Tappi J., 80:181 (1997). Wood strength, density, and grain slope in wet wood and standing trees can be determined by measuring the visible and near infrared spectral data in conjunction with multivariate analysis. See, U.S. Patent Application Publication Nos. 2002/0107644 and 2002/0113212. Lumen size can be measured using scanning electron microscopy. Lignin structure and chemical properties can be observed using nuclear magnetic resonance spectroscopy as described in Marita et al., J. Chem. Soc., Perkin Trans. I 2939 (2001).

The biochemical characteristic of lignin, cellulose, carbohydrates and other plant extracts can be evaluated by any standard analytical method known including spectrophotometry, fluorescence spectroscopy, HPLC, mass spectroscopy, and tissue staining methods.

It will be readily apparent to one of ordinary skill in the relevant arts that other suitable modifications and adaptations to the methods and applications described herein can be made without departing from the scope of the invention or any embodiment thereof.

The following examples are set forth as representative of specific and preferred embodiments of the present invention. These examples are not to be construed as limiting the scope of the invention in any manner. It should be understood that many variations and modifications can be made while remaining within the spirit and scope of the invention.

### EXAMPLES

### Example 1

### Plant Materials

Commercial E. urophylla and E. saligna clones were provided by International Paper, USA and were received as shoot clumps on solid medium Shoot clumps were divided as needed and maintained as stock culture. The clones were maintained as shoot clumps in Magenta boxes and subcultured every 4-6 weeks. The clumps were transferred to Euc Maintenance medium (Table 1) with BA at 0.25-0.5 mg/l in Magenta boxes. The cultures were grown under full light conditions at an intensity of about 120 μE/m²/s with a photoperiod of 16 hours, and the temperature of the growth room is 21° C., and clumps were maintained at 4 clumps per Magenta box. The preferred explant are tissues with advanced vascular tissues, such as internodes, petioles and midribs.

### Example 2

### Construct

Two constructs were used for the transformation experiments: pWVZ20 and pWVR133. Binary plasmid pWVZ20 contains an herbicide resistant gene driven by a plant promoter and the β-glucuronidase (GUS) gene driven by a constitutive promoter between T-DNA borders. Binary plasmid pWVR133 contains the same herbicide resistant gene construct, but the GUS gene contains an intron and is driven by an actin promoter. GUS expression of pWVR133 only occurs in plant cells but not in bacterial cells. The Agrobacterium strain GV2260 (Deblaere et al. 1985) was used for the transformation studies. One of ordinary skill in the art would be able to use any binary construct and any similar Agrobacterium strain having an herbicide resistant gene operably linked to a constitutive plant promoter.

### Example 3

### Preparation of Agrobacterium for Transformation

Agrobacterium containing either pWVZ20 or pWVR133 were grown on YEP medium (10 g/l yeast extract, 10 g/l peptone and 50 mg/l NaCl, pH 7.0-7.2) for 3 days. A single colony from the plate was selected and grown in 20-50 ml liquid YEP medium with kanamycin at 100 mg/l and rifampicin 50 mg/l. Cultures were incubated overnight in a shaking incubator at 28° C. and 150 rpm. The over night culture with an OD of about 0.6-1.1 was spun down in a desktop centrifuge at 3000 g for 20 minutes and resuspended with Agrobacterium Induction Medium or AIM (WPM salts with glucose at 5 g/l, 250 μM acetosyringone, 2 mM phosphate buffer, and 0.05 M MES, pH 5.8) with an OD of about 0.6-1.1. Cultures were incubated for 25 minutes at 28° C. before infection. Bacterial concentration was determined before and after infection.

### Example 4

### Preparation of Explants for Infection and Pre-Culture

Eucalyptus stock cultures maintained on Euc Maintenance medium were used as the sources of the explants. Although leaves, petioles, internodes, floral tissues, and embryogenic tissues can be used for transformation, leaf explants were selected because leaves are abundant. Healthy and newly opened leaves were selected for transformation. The tip portions of the leaves were removed by scissors or forceps to increase the number of wounded cells. Explants were placed on pre-culture medium (Table 2) abaxial side down. The pre-culture medium is a nutrient medium upon which plant explants are cultured before Agrobacterium transformation. Specifically, the inventive pre-culture medium increases transformation efficiency and plant regeneration. While the present pre-culture medium comprises acetosyringone, other Agrobacterium inducers may be used. Woody Plant Medium (WPM) salts (Loyd and McCown, 1980) were used in the present pre-culture medium; however, other salt media, such as MS medium (Murashige and Skoog 1962) or Lepoivre medium, may be used. Optionally, the pre-culture medium may comprise plant growth regulators, including auxin and cytokinin.

In the present invention, plant explants were pre-cultured for four days in the dark on the pre-culture medium displayed in Table 2. While not required, the instant pre-culture medium contained both auxin and cytokinin. Additionally, plant explants may be cultured on the pre-culture medium from one to six days before Agrobacterium transformation.

### Example 5

### Agrobacterium Inoculation and Removal

Induced Agrobacterium culture was prepared as described in Example 3 and the culture was dripped onto each explant by pipette. Sufficient Agrobacterium culture was dripped to ensure that all the cut edges were covered with bacterial solution. Alternatively, the explants may be transformed by vacuum infiltration, floral dip, and other methods of Agrobacterium-mediated transformation. Following transformation, explants covered with Agrobacterium culture were placed in the dark for four days of co-cultivation. Alternatively, the explants may be co-cultivated with Agrobacterium under light conditions. Additionally, the explants may be co-cultivated with Agrobacterium under light or dark conditions for 2-10 days, preferably 4 days. Following co-cultivation, the explants were transferred to Euc Regeneration medium (Table 3) with 400 mg/l timentin. There is no need to wash explants. Explants were cultured on this medium for four days before transfer to a selection medium. In the present example, the selection medium is the Euc Regeneration medium supplemented with both timentin and an herbicide selection agent.

### Example 6

### Regeneration of Transgenic Shoots

Shoot clumps that survive selection are maintained on Euc regeneration medium containing herbicide and timentin, and they are transferred every 3 weeks until shoots proliferate and initially elongate. For transformation experiments with pWVR133, leaf and stem tissues from the regenerated shoots are stained for GUS expression as soon as the shoots are developed. For transformation experiments with pWVZ20, leaf and stem tissues from the regenerated shoots are stained for GUS expression when the shoots are further developed and free from residual Agrobacterium.

### Example 7

### GUS Staining

GUS staining was performed to monitor the frequency of Agrobacterium infection and to ensure that the selected shoots are not escapes or chimeras. For transformation experiments with pWVR133, the leaf and stem tissues from the regenerated shoots were stained for GUS expression immediately upon shoot development. For transformation experiments with pWVZ20, leaf and stem tissues from the regenerated shoots were stained for GUS expression when shoots are further developed and free from residual Agrobacterium. To determine GUS activity, the explants were incubated in a substrate comprising 100 mM phosphate buffer (pH 7.0), 0.05% dimethyl suphoxide, 0.05% Triton X-100, 10 mM EDTA, 0.5 mM potassium ferrocyanide, and 1.5 mg/ml 5-bromo-4-chloro-3-indolyl-β-D-glucuronide (X-gluc). The explants were subjected to 10 minutes of vacuum before an overnight incubation at 37° C. Following overnight incubation, GUS foci were counted.

### Example 8

### RNA Isolation

RNA was isolated from leaf and shoot tissues of wild-type and transformed E. urophylla with RNAqueous™-96 kit, according to the manufacturer's instructions. Briefly, 0.1 to 1.5 mg tissue was suspended in 300 μl Lysis/Binding buffer and ground to a fine powder with a plastic pestle. The samples were centrifuged at maximum speed for 3 minutes and the supernatant was transferred to a fresh plate. Three hundred μl (1×v/v) 64% ethanol was added to the supernatant and the samples were vortexed briefly. The samples were then centrifuged for 2 minutes at 1850 g. Following centrifugation, an optional DNase treatment step was performed. To each sample, 5 μl DNase+35 μl DNase buffer was added to the center of the filter pad and the samples were incubated at room temperature for 20 minutes. After the 20 minute incubation, 600 ml Wash Buffer A was added to each sample and the samples were centrifuged at maximum speed for 2 minutes. Following centrifugation, the supernatants were discarded and 600 μl Wash Buffer B was added to the filter pad of each sample. Following centrifugation at maximum speed for 2 minutes, the supernatants were discarded and 600 μl Wash Buffer C/D was added to each filter pad. The samples were centrifuged at maximum speed for 2 minutes and the supernatants were discarded. Following a second rinse with Wash Buffer C/D, 50 μl RNA elution solution was added and the samples were centrifuged at maximum speed for 2-3 minutes. The RNA elution step was repeated and the eluted RNA samples were stored at −80 degrees Celsius until further use.

### Example 9

### RT-PCR

To determine the presence and expression of the ALS herbicide resistance gene, RT-PCR was performed. Total RNA was isolated, as described in Example 8, from transformed and wild-type samples of E. urophylla plants. Following RNA isolation and quantitation, RT-PCR was performed with Ready to Go RT-PCR kit (Pharmacia), according to the manufacturer's instructions. Generally, 5 μl total RNA (about 20 ng RNA) was added to a 45 μl RT-PCR reaction mixture containing 1 μl Oligo dTn, 1 μl 3 μM ALS forward primer, 1 μl 3 μM ALS reverse primer, and 42 μl water.

To denature the RNA, the tubes were heated to a temperature of at least 75° C. for 3 minutes. The samples were then incubated at 42° C. for 30 minutes. Following incubation, RT-PCR was performed as follows:

| | |
|---|---|
| Cycle 1: | 95° C. for 5 minutes |
| | 55° C. for 1 minute |
| | 72° C. for 1 minute |
| Cycles 2-30 | 95° C. for 1 minute |
| | 55° C. for 1 minute |
| | 72° C. for 1 minute |
| Cycle 31 | 95° C. for 1 minute |
| | 55° C. for 1 minute |
| | 72° C. for 10 minutes |

Following completion of RT-PCR, the PCR amplification products were visualized on an agarose gel stained with ethidium bromide.

### Example 10

### Preparation of Vectors with Selectable Markers ALS or ASA2

DNA vectors were designed that contain a reporter gene and the nptII selectable marker as well as the alternative selectable markers als or asa2 driven by constitutive promoters. The relative positions of the genes in the vectors were designed such that in a binary vector, the selectable marker nptII would be near the border that is most often lost during transformation, and the alternative selectable marker would be between the nptII gene and the uidA reporter gene. This was done in order to allow for selection experiments in which the cells could be selected first or in parallel using nptII, and the presence of the nptII gene, as evidenced by successful geneticin selection, is evidence of a high likelihood that the alternative selectable marker linked between these two genes is present. This configuration facilitated testing and development of the selection system using rapid non-molecular measures of transformation success until alternative marker selection conditions were established. The vectors were designed so that convenient restriction sites allow the subsequent removal of the nptII and uidA genes, as well as the insertion of other genes of interest. The alternative selectable marker, positioned near the border that is most often lost during transformation, allows genes of interest to be co-transferred with the alternative selectable marker.

The als gene is publicly known and available. GenBank contains numerous als genes from plants. For example, GenBank Accessions Nos. gi 30693053, gi 30685789, and gi 30685785 provide als genes isolated from Arabidopsis thaliana. While any als sequence may be used, the present invention used an als cassette subcloned, using SalI and SmaI, from pML1 (DuPont™). The als cassette was then cloned into the vector Bluescript™ (Stratagene) to make the construct pWVC20. (FIG. 1) The cassette was placed under the expression of a ubiquitin promoter subcloned using the restriction enzymes HindIII and NcoI from p2554 (Norris et al. 1993. Plant Molecular Biology, 21:895-906) to make pWVC22 and provide additional restriction sites and allow for high copy accumulation. The larger (4.9 kb) als plant expression cassette, restricted with ApaI and NotI, was then inserted into binary vectors containing the nptII and uidA genes driven by different constitutive promoters, such that the als plant expression cassette is located between the nptII and uidA genes and flanked by convenient restriction sites, to give rise to pWVC23 (FIG. 3) and pWVC24 (FIG. 4). Thus, it is straightforward to obtain pWVC25 (FIG. 5a), containing only the uidA and als plant expression cassettes, and pWVC26 (FIG. 5b), containing only the als plant expression cassette, into which cassettes of interest can be cloned for insertion into plants using als selection. Such cassettes of interest can include, but are not limited to, genes that will add or increase the production of molecules of interest, such as syringyl lignin biosynthesis genes or stress resistance genes or RNAi or antisense constructs that decrease the production of molecules of interest.

The asa2 gene is publicly known and available. GenBank contains numerous asa2 genes from plants. For example, GenBank Accessions Nos. gi 4256949 and gi 3348125 provide asa2 genes isolated from plants. While any asa2 sequence may be used, the present invention used an asa2 gene subcloned from the plasmid pUC35SASA2 (Song, H. S. et al. (1998) Plant Physiol. 117:2:533-548) using the restriction enzymes SphI and BamHI to give a 4800 bp promoterless fragment for pWVC30 (FIG. 6) and was placed under the expression control of a ubiquitin promoter subcloned from p2554. This T4-ligated cassette was cloned into the Bluescript™ vector pBSKII+ to provide two additional restriction sites, ApaI and NotI. These restriction enzymes were used to cut out a 3800 bp fragment, which was ligated into a binary vector containing the nptII and uidA genes driven by different constitutive promoters, as described above, to produce pWVC33 (FIG. 7). The asa2 plant expression cassette is located between the nptII and uidA genes and flanked by additional convenient restriction sites.

Thus, it is straightforward to obtain pWVC34 (FIG. 8a), containing only the uidA and asa2 plant expression cassettes, and pWVC35 (FIG. 8b), containing only the asa2 plant expression cassette, into which cassettes of interest may be cloned for insertion into plants using asa2 selection. Such cassettes of interest include, but are not limited to, genes that will add or increase the production of molecules of interest, such as syringyl lignin biosynthesis genes or stress resistance genes or RNAi or antisense constructs that decrease the production of molecules of interest.

The pWVC23 (FIG. 3), pWVC24 (FIG. 4) and pWVC33 (FIG. 7) plasmids were transformed into the Escherechia coli XL10-Gold bacterial competent cell line and the Agrobacterium tumefaciens cell line GV2260, using standard techniques.

### Example 11

### Method for Increasing Infection Rate

This example teaches a method for increasing Agrobacterium infection rate. In particular, this example teaches a method that stimulates cell growth, increases the number of target cells, and promotes infection. Explants from E. urophylla and E. saligna were prepared as described in Example 1 and Agrobacterium strain GV2260 containing pWVR133 was prepared as described in Example 3. Harvested explants were incubated in the dark for 4 days either on Eucalyptus regeneration medium or a medium with various auxin-rich growth regulators. Agrobacterium culture was dripped on explants and co-cultured with the explants on the same medium for 3 days in dark. Then, explants were removed from the Agrobacterium puddle and transferred to Euc Regeneration medium supplemented with 400 mg/l timentin for 4 days. Following transfer to regeneration medium, the explants were stained for GUS expression. See Table 5 below. As shown in Tables 6-7, various auxin-type growth regulators had a positive effect on GUS expression 7 days post infection.

TABLE 5
Infection of E. urophylla and E. saligna clones with p35SGUS intron
(35S::GUSIN, NOS::NPTII)

| | | | | | | Avg. No. |
|---|---|---|---|---|---|---|
| | | | | | No. | Foci/ |
| | | No. | No. | % | GUS | Responding |
| Clone | Species | Explants | GUS+ | GUS+ | Foci | Explant |
| IPB10 | E. | 20 | 2 | 10 | 2 | 1.0 |
| | urophylla | | | | | |
| IPB13 | E. saligna | 20 | 19 | 95 | 162 | 8.5 |

TABLE 6
Auxin Pre-treatment Increases the Agrobacterium Infection
Rate for an Elite Eucalyptus urophylla clone

| Pre-treatment | | | | | |
|---|---|---|---|---|---|
| Medium | | | | | Avg. No. |
| (EucReg | | | | | Foci/ |
| Medium + | No. | No. | % | No. GUS | Responding |
| PGR in mg/l) | Explants | GUS+ | GUS+ | Foci | Explant |
| 5.0 zeatin/ | 25 | 3 | 12 | 6 | 2.3 |
| 0.1 NAA | | | | | |
| (Control | | | | | |
| Regeneration | | | | | |
| Medium) | | | | | |
| 0.25 2,4-D/ | 24 | 4 | 17 | 7 | 1.5 |
| 0.5 zeatin | | | | | |
| 1.0 2,4-D/ | 25 | 15 | 60 | 155 | 10.3 |
| 0.5 zeatin | | | | | |

Although there is a significant increase in infection rate and the number of cells expressing GUS, the control shows a delay of shoot regeneration from medium with 1.0 mg/l 2,4-D. The zeatin level is raised to 5 mg/l, the same level as the level in regeneration medium

TABLE 7
Various Auxin-type Growth Regulators Show Positive Influence
on Infection of an Elite Eucalyptus urophylla Clone

| Pre-treatment | | | | | |
|---|---|---|---|---|---|
| Medium | | | | | Avg. No. |
| (EucReg Medium + | | | | No. | Foci/ |
| 5 mg/l zeatin + | No. | No. | % | GUS | Responding |
| auxins in mg/l) | Explants | GUS+ | GUS+ | Foci | Explant |
| 0.1 NAA | 25 | 4 | 16 | 5 | 1.3 |
| (Control | | | | | |
| Regeneration | | | | | |
| Medium) | | | | | |
| 1.0 2,4-D | 25 | 12 | 48 | 64 | 5.3 |
| 0.5 2,4-D | 25 | 20 | 80 | 97 | 4.9 |
| 2.0 NAA | 25 | 9 | 36 | 30 | 3.3 |
| 1.0 NAA | 25 | 13 | 52 | 35 | 2.7 |
| 3.0 IAA | 48 | 46 | 96 | 565 | 12.3 |
| 2.0 IAA | 25 | 16 | 64 | 167 | 10.4 |
| 1.0 IAA | 25 | 16 | 64 | 116 | 7.3 |
| 1.0 TDZ | 25 | 0 | 0 | 0 | 0.0 |
| 0.5 TDZ | 25 | 1 | 4 | 3 | 3.0 |
| 0.1 TDZ | 25 | 3 | 12 | 25 | 8.3 |

The results from experiments above show that pre-culture of explants with medium rich in various auxin-type plant growth regulators increases the infection rate as evaluated by the frequency of explants with GUS foci and the average number of GUS foci per responding explant. In addition, the regeneration is not affected by the pre-culture in control experiments.

### Example 12

### i Agrobacterium Inducer Improves Infection Rate

The results indicate that AS alone in the pre-culture medium stimulates infection. In addition, GUS staining indicates that AS increases the infection of the wounded cells along the cut edges, where transgenic plants normally arise.

### Example 13

### Combining Agrobacterium Inducer and Growth Regulator Improves Infection Rate

This example indicates that the combination of AS and auxins in the pre-culture phase could increase the infection rate even higher. In the following examples, explants from E. urophylla clones were cultured on auxin-rich pre-culture medium with or without 250 μM AS before infection. Explants were pre-cultured with Euc Regeneration medium as the control. Following pre-culture, the explants were inoculated with Agrobacterium as described in Example 5.

TABLE 8
Effect of combining auxin with Agrobacterium inducer
on E. urophylla infection

| Pre-culture | | | | | |
|---|---|---|---|---|---|
| Medium | | | | | Avg. No. |
| (EucReg | | | | No. | Foci/ |
| Medium + | No. | No. | % | GUS | Responding |
| PGR in mg/l) | Explants | GUS+ | GUS+ | Foci | Explant |
| 5.0 zeatin/0.1 NAA | 50 | 11 | 22 | 37 | 3.4 |
| (Control | | | | | |
| Regeneration | | | | | |
| Medium | | | | | |
| 5.0 zeatin/ | 50 | 36 | 72 | 388 | 10.8 |
| 0.25 2,4-D | | | | | |
| 5.0 zeatin/ | 50 | 42 | 84 | 754 | 18.0 |
| 0.25 2,4-D +AS | | | | | |
| 5.0 zeatin/2 IAA | 49 | 36 | 73 | 289 | 8.0 |
| 5.0 zeatin/ | 49 | 47 | 96 | 855 | 18.2 |
| 2 IAA +AS | | | | | |

These results show a consistent beneficial effect on infection across a range of clones by using both AS and auxin stimulation in the pre-culture medium. The clones have an increase in both the infection frequency and the average number of GUS foci per responding explant. In addition, the infection of wounded cells along the cutting edge significantly increases.

### Example 14

### RT-PCR to Assess Expression of Herbicide Tolerance Gene

This example shows the presence and expression of a herbicide tolerance gene in a sample of transgenic lines. As described in Example 8, RNA was isolated from the leaves and stem tissues of each putative transgenic line. In particular, RNA was isolated from transformed E. urophylla lines. In addition, RNA was isolated from wild-type E. urophylla. For each line, a 5 μl RNA sample was used for RT-PCR to assess the presence and the expression of a herbicide tolerance gene. RT-PCR was performed with the Ready-to-Go RT-PCR kit (Pharmacia), according to the manufacturer's instructions. Briefly, each 5 μl RNA preparation was added to separate RT-PCR reactions containing oligo(dT)ₙ and herbicide-resistant gene primers. Following RT-PCR, the amplification products were visualized on an ethidium bromide-stained agarose gel.

Agarose gel electrophoresis of the RT-PCR products revealed that all of the tested transgenic E. urophylla lines express the herbicide tolerance gene, while the control plants do not express ALS.

### Example 15

This example demonstrates the adaptability of the present transformation methodology to a different selectable marker, neomycin phosphotransferase (NPTII) gene, which confers resistance to aminoglycoside antibiotics neomycin, kanamycin or geneticin (G418). E. urophylla leaf explants were infected with Agrobacterium strain GV2260 harboring pWVC33, which contains GUS gene (driven by an constitutive promoter) and NPTII gene (driven by a different constitutive promoter). Before transforming, the explants were pre-cultured for 4 days on a medium having auxin enrichment (IAA at 2 mg/l instead of 0.1 mg/l NAA in standard regeneration medium) and acetosyringone (250 μM). As described in Example 6, the explants were infected with Agrobacterium, co-cultivated on the same medium for 3 days, and then transferred to a regeneration medium supplemented with 400 mg/l timentin for 4 days. Then, the explants were transferred to a selection medium containing geneticin instead of an herbicide selection agent.

The transformants were placed on geneticin selection medium containing geneticin (either 20 or 30 mg/l). Instead of the 3- or 4-week transfer cycles used with an herbicide selection medium, the explants were transferred bi-weekly to fresh geneticin medium. After about 10 weeks of growth on geneticin selection medium, samples of geneticin-resistant calli were collected and stained with x-gluc for GUS expression, as described in Example 7. The following data summarizes GUS expression in geneticin-selected transformants.

TABLE 9
Transformation of E. urophylla with antibiotic selectable marker (NPTII)

| Concentration | | | | |
|---|---|---|---|---|
| of Geneticin | Light Conditions | No. | No. Calli | No. GUS⁺ |
| (mg/l)/ | for cultures | Explants | Stained | Calli |
| 20 | Regular shaded | 162 | 5 | 0 |
| | light | | | |
| 30 | Regular shaded | 162 | 17 | 13 |
| | light | | | |
| 30 | Dark | 162 | 6 | 4 |

### Example 16

Based on the data from the above examples, E. urophylla clones can be transformed with a foreign DNA by the disclosed method. For example, elite E. urophylla clones can be transformed with a gene that encodes an enzyme involved in cellulose synthesis, such as a cellulose synthase. Cellulose synthase binds UDP-glucose and transfer the sugar to the non-reducing end of the nascent glucan chain. Using the methods of the present invention, the UDP-glucose binding domain can be overexpressed in a transgenic plant.

The transformation of Eucalyptus elite clones with a sense UDP-glucose binding domain sequence operably-linked to a constitutive promoter confers an enhanced growth phenotype, as evidenced by increases in cellulose synthesis, wood density, and tensile strength. Leaf explants are harvested from stock Eucalyptus plants and the explants are cultured on a pre-culture medium. The pre-culture medium comprises auxin, cytokinin, and an Agrobacterium inducer, such as acetosyringone, to stimulate cell division along the excised edges of the tissue explant. Following four days of pre-culture, the explants were inoculated with Agrobacterium strain GV2260 containing a plasmid bearing a portion of the GS coding region operably linked to the constitutive promoter. The explants were co-cultivated for 3 days before transfer to Euc Regeneration medium. The explants were cultured on Euc Regeneration medium for 4 days before transfer to selection medium containing an herbicide.

Following the selection of herbicide-resistant transformants, the transformants were assayed for GUS expression. Upon the confirmation of GUS expression, shoots were harvested and transferred to a rooting medium. The rooting medium comprises BTM-1 salts supplemented with 5 g/l activated carbon, and rooting development usually occurs after 2-4 weeks. Upon development of the primary root system, the transformed plants are transferred to soil.

What is claimed is:

1. A method for transforming at least one cell of a E. urophylla explant with a foreign DNA, comprising
(i) culturing a tree explant on a pre-culture medium comprising an inducer of Agrobacterium;
(ii) exposing said explant to an Agrobacterium strain containing a transformation vector carrying said foreign DNA;
(iii) selecting a transformed explant, wherein said foreign DNA is transferred to at least one cell of said transformed explant; and
(iv) regenerating said transformed explant to produce a complete plant.

2. The method of claim 1, wherein said inducer of Agrobacterium is acetosyringone.

3. The method of claim 2, wherein the concentration of said acetosyringone is from about 10 to about 400 mg/l.

4. The method of claim 3, wherein the concentration of said acetosyringone is from about 5 to about 200 mg/l.

5. The method of claim 1, wherein said medium further comprises auxin.

6. The method of claim 1, wherein said medium further comprises cytokinin.

7. The method of claim 1, wherein said explant is pre-cultured in the dark from about 1 to about 6 days.

8. The method of claim 1, wherein said explant is pre-cultured for about 4 days.

9. The method of claim 5, wherein said auxin is selected from the group consisting of NAA, 2,4-D, IBA, and IAA.

10. The method of claim 9, wherein the concentration range of any one of NAA, 2,4-D, IBA, and IAA is from about 0.1 to about 10 mg/l.

11. The method of claim 10, wherein said concentration range is from about 0.2 to about 5 mg/l.

12. The method of claim 11, wherein said concentration range is from about 0.2 to about 3 mg/l.

13. The method of claim 6, wherein said cytokinin is selected from the group consisting of zeatin, kinetin, and BA.

14. The method of claim 13, wherein the concentration range of any one of said zeatin, kinetin, and BA is from about 0.25 to about 15 mg/l.

15. The method of claim 14, wherein said concentration range is from about 1 to about 10 mg/l.

16. The method of claim 15, wherein said concentration range is from about 1 to about 6 mg/l.

17. The method of claim 1, wherein said explant is at least one of a leaf, a petiole, an internodal tissue, a floral tissue, or an embryogenic tissue.

18. The method of claim 1, wherein said tissues are selected independent of age or developmental stage.

19. The method of claim 1, wherein said method is genotype-independent.

20. The method of claim 1, wherein all of the cells of said transformed explant comprise said foreign DNA.

21. A method for producing a non-chimaeric E. urophylla tree, comprising
(i) pre-culturing a E. urophylla explant on a medium comprising an inducer of Agrobacterium;
(ii) exposing said explant to an Agrobacterium strain harboring a vector capable of transferring a gene to a plant cell;
(iii) selecting a transformed explant; and
(iv) growing said explant to produce a non-chimaeric tree.

22. A method for transforming at least one cell of a E. saligna explant with a foreign DNA, comprising
(i) culturing a tree explant on a pre-culture medium comprising an inducer of Agrobacterium;
(ii) exposing said explant to an Agrobacterium strain containing a transformation vector carrying said foreign DNA;
(iii) selecting a transformed explant, wherein said foreign DNA is transferred to at least one cell of said transformed explant; and
(iv) regenerating said transformed explant to produce a complete plant.

23. The method of claim 22, wherein said inducer of Agrobacterium is acetosyringone.

24. The method of claim 23, wherein the concentration of said acetosyringone is from about 10 to about 400 mg/l.

25. The method of claim 24, wherein the concentration of said acetosyringone is from about 5 to about 200 mg/l.

26. The method of claim 22, wherein said medium further comprises auxin.

27. The method of claim 22, wherein said medium further comprises cytokinin.

28. The method of claim 22, wherein said explant is pre-cultured in the dark from about 1 to about 6 days.

29. The method of claim 22, wherein said explant is pre-cultured for about 4 days.

30. The method of claim 26, wherein said auxin is selected from the group consisting of NAA, 2,4-D, IBA, and IAA.

31. The method of claim 30, wherein the concentration range of any one of NAA, 2,4-D, IBA, and IAA is from about 0.1 to about 10 mg/l.

32. The method of claim 31, wherein said concentration range is from about 0.2 to about 5 mg/l.

33. The method of claim 32, wherein said concentration range is from about 0.2 to about 3 mg/l.

34. The method of claim 27, wherein said cytokinin is selected from the group consisting of zeatin, kinetin, and BA.

35. The method of claim 27, wherein the concentration range of any one of said zeatin, kinetin, and BA is from about 0.25 to about 15 mg/l.

36. The method of claim 35, wherein said concentration range is from about 1 to about 10 mg/l.

37. The method of claim 36, wherein said concentration range is from about 1 to about 6 mg/l.

38. The method of claim 22, wherein said explant is at least one of a leaf, a petiole, an internodal tissue, a floral tissue, or an embryogenic tissue.

39. The method of claim 22, wherein said tissues are selected independent of age or developmental stage.

40. The method of claim 22, wherein said method is genotype-independent.

41. The method of claim 22, wherein all of the cells of said transformed explant comprise said foreign DNA.
